(19) United States
(12) Patent Application Publication
(10) Pub. No.: US 2011/0262546 A1
(43) Pub. Date: Oct. 27, 2011

(54) GOLD NANOPARTICLES COATED WITH POLYELECTROLYTES AND ALBUMIN

(76) Inventors: Giuseppe Antonio Legname, Trieste (IT); Silke Krol, Trieste (IT); Maria Fernanda Costa De Sousa, Trieste ( (IT)

(21) Appl. No.: 13/127,904

(22) PCT Filed: Nov. 5, 2009
(86) PCT No.: PCT/IB2009/054922

§ 371 Date: Jun. 27, 2011

(30) Foreign Application Priority Data
Nov. 7, 2008 (IT) RM2008AQ000602

(51) Int. Cl. A61K 9/50; A61P 25/28; A61P 25/14; A61P 25/00; A61K 31/785; A61P 25/16; B82Y 40/00; B82Y 5/00

(52) U.S. Cl. 424/491; 424/78.35; 427/2.14; 977/773; 977/906; 977/890

(57) ABSTRACT

It is described a gold nanoparticle coated with from two to five layers of a combination of a polyelectrolyte having amino functionality and a polyelectrolyte having sulfonic functionality, or with one single layer of said polyelectrolyte having amino functionality, preferably polyallylamine, or sulfonic functionality, preferably polystyrenesulfonic, characterized in that said nanoparticle comprises an outer layer of albumin. It is also described the process for its preparation, its use as carriers intended to cross blood-brain barrier and its use as medicament, in particular for treatment of neurodegenerative diseases, more in particular of diseases caused by protein aggregates, such as prion diseases, Alzheimer's disease, Parkinson's disease, Huntington's disease and amyotrophic lateral sclerosis. Also described are pharmaceutical compositions comprising said nanoparticle.

The present invention refers to the medical field and relates in particular to gold nanoparticles coated with polyelectrolytes for use as medicaments, particularly for treatment of neurodegenerative diseases.

### BACKGROUND OF THE INVENTION

Neurodegenerative diseases such as Alzheimer's disease, Parkinson's disease, Huntington's disease, amyotrophic lateral sclerosis (ALS) and prion diseases are all characterized by the accumulation of protein aggregates in the nervous central system probably involved in their pathogenesis. In the case of Alzheimer's disease, the most common neurodegenerative disease with a national incidence of more than 800,000 patients and with a presence of more than 26 million patients in the world, it is characterized by deposits of Aβ in the plaques called amyloid and of neurofibrillary tangles composed mostly by tau phosphorylated protein. In the case of Parkinson's disease, the second neurodegenerative disease for higher incidence, the so-called Lewy's bodies are composed by aggregates of amyloid nature of the alpha-synuclein protein. For prion diseases, the aggregates are composed predominantly of prion protein. These diseases, such as the transmissible spongiform encephalopathies, include Creutzfeldt-Jakob disease (CJD) in humans, scrapie and bovine spongiform encephalopathy (BSE) in animals. These neurodegenerative diseases are incurable and fatal and are associated with neuronal cell death, the characteristic “spongiform” vacuolation of brain tissue and the accumulation of the isoform associated with prion protein disease expressed by endogenous way (Prusiner S B. Shattuck; Lecture—Neurodegenerative Diseases and Prions. N. Engl. J. Med. 2001 May 17; 344(20):1516-26. Review).

The central feature of prion diseases is the accumulation in the brain and in certain other tissue of the disease associated PrP^{Sc} protein, which derives from the cellular protein form encoded by the host PrP^{C}. Although the function of this protein is still unknown, PrP^{C} is involved in the pathogenesis of the prion, for example, the presence in genetic cases of prion diseases of mutations of the coding sequence of the human prion protein (PRNP) gene, resulting in hereditary forms of prion disease (Jackson J S, Collinge J, J. Clin. Pathol: Mol. Pathol.; 2001; 54:393-399), and the presence of PrP^{C} is necessary for prion propagation and development of prion disease (Bueler et al., 1993). PrP^{Sc} derives from PrP^{C} by post-translational conformational modification (Borchelt et al., 1990; Caughey and Raymond, 1991) and is extracted from diseased brain tissue as an aggregate material, which is distinguished from PrP^{C} by its partial resistance to protease digest and insolubility in detergents. An abundance of evidence now supports the hypothesis of the “single protein” (Griffith, 1967; Prusiner, 1982), which states that the PrP^{Sc} is the major constituent, or the only, transmissible agent or prion (Bolton et al., 1982) and acts as conformational template to promote conversion of endogenous PrP^{C} to PrP^{Sc} (for a review see Prusiner, 2001). The conversion mechanism and the structure of the infective agent are still unclear.

At molecular level, the therapies for prion disease may be directed to PrP^{C}, to PrP^{Sc} or the conversion process between the two isoforms of the prion protein. A therapy directed against PrP^{Sc}, the isoform associated with the disease, may seem the more logical approach, but may have no effect in the progression of the disease or might even extend its life, if PrP^{Sc} is a non-pathological point of arrival of the pathogenic conversion process, or if the depositing rate of PrP^{Sc} is critical for disease progression.

A recent review of attempts to find a therapy for prion diseases is given in Trevitt and Collinge Brain, 2006, 129, 2241-2265, to which reference is made, including the citations reported there.

The patent application DE 10 2004 040 119 describes the use of nanoparticles in treatment of prion infections. In particular, the reference describes colloidal systems based on gold or silver, whose particles have a preferred size of about 5 nm, but it is also specified a maximum size of 20 nm. The particles must have a surface charge, for example given by the colloidal system. In a totally generic way, and without providing practical examples, the reference also mentions possible metallic “clusters”, non metallic compounds, such as borates, silicates, polyoxometalates, organic complexes with transition metals, nanoparticles with organic compounds, for example polycyclic aromatic hydrocarbons, fullerene, macrocyclic compounds, dendrimers. This reference indicates as a critical factor for the efficacy against prion fibers the ionic strength of the environment.

In literature, it is known that sulfate groups bind selectively to prion fibers, but are not able to dissolve them (Trevitt C. & Collinge J. (2006) Brain, 129, 2241-2265) while the primary amino groups are able to dissolve the prion fibers and to eliminate them from cells (Supattapone S. et al. (2001) J. Virology, 75, 3453-3461).

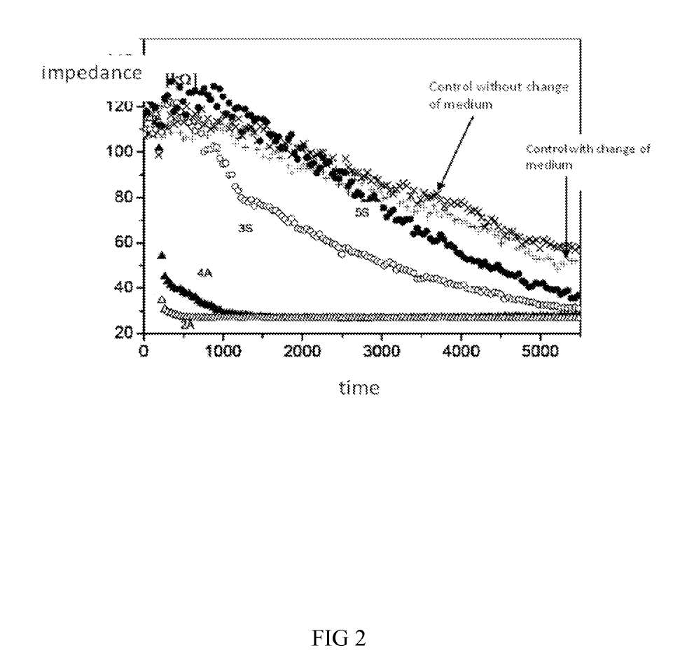

However, primary amines can not be used as such in a subject affected by prion disease because of their toxicity, particularly for cells of the blood-brain barrier, which in the case of the present invention is an absolutely critical element for the administration of a drug for the treatment of neurodegenerative diseases. The toxicity of primary amines towards cells of the blood-brain barrier is described in Chanana et al. Nano Letters (2005) 5(12), 2605-2612, see in particular FIG. 2 in this description, and by other authors (Boussif, O.; Delair, T.; Brua, C.; Veron, L.; Pavirani, A.; Kolbe, H. V. J., Synthesis of Polyallylamine Derivatives and Their Use as Gene Transfer Vectors in Vitro. Bioconjugate Chem. 1999, 10, 877-883 e Clare R Trevitt and John Collinge: A systematic review of prion therapeutics in experimental models. Brain (2006), 129, 2241-2265). According to the work of Chanana et al., cytotoxicity is highly dependent on the number of layers and surface charge in the case of nanoparticles coated with multilayers of polyelectrolyte. The polycations are more toxic and are precisely the most promising molecules in the activity against prion aggregates. The toxicity is inversely proportional to the number of polyelectrolyte layers.

In view of these results, the use of molecules carrying primary amino groups to dissolve the prion fibers, and to be used more generally in relation to protein deposits typical of neurodegenerative diseases appears prohibitive.

Molecules like GAGs (Glycosaminoglycans) carrying both amino and sulfate functionalities are not able to stop the progression of the disease (Trevitt and Collinge Brain, 2006, 129, 2241-2265).

Schneider and Decher (Nano Letters, 2004, Vol. 4, No. 10, 1833-1839) describe the method of deposition layer by layer (LBL) of polyelectrolytes on gold nanospheres, obtaining stable nanoparticles.

Dorris et al. (Langmuir, 2008, 24(6), 2532-2538) study the stabilization of gold nanoparticles, stabilized with 4-(dimethylamino)pyridine (DMAP), coated with sodium poly(4-styrenesulfonate), through electrostatic self assembly. This work also explores the effect on the stability of nanoparticle by polyallylamine hydrochloride (PAH).

Schneider and Decher (Langmuir, 2008, 24, 1778-1789) study the parameters that affect the stability of the above systems in the preceding works.

The present invention intends to solve the problem of toxicity of primary amine, particularly towards the cells of blood-brain barrier, thus providing an effective means for therapy of prion diseases.

### SUMMARY OF THE INVENTION

It has now been found that a polyelectrolyte having amino functionality co-absorbed with a polyelectrolyte having sulfonic functionality on a gold nanoparticle and with an outer layer of albumin are endowed with the desired activity against prion fiber, but the primary amine toxicity is substantially decreased or lost.

In particular it has been found that primary amines co-absorbed together with albumin on a negatively coated (PSS) gold nanoparticle system maintain their desired activity against prion fiber but substantially loose or decrease their cytotoxicity. It has also surprisingly been found that the nanoparticle with primary amines as outermost layer, hence with a positive net charge, shows an inhibition power on prion protein aggregation, and in general of the protein which causes the neurodegenerative disease, markedly higher than that known in the literature, wherein it is reported a greater effect of the nanoparticle with a negative net charge, for example with polysulfonate (J. Mol. Biol. 2007 Jun. 15; 369(4):1001-14. Thioaptamer interactions with prion proteins: sequence-specific and non-specific binding sites. King D J, Safar J G, Legname G, Prusiner S B).

Therefore it is an object of the present invention a gold nanoparticle coated with two to five layers of a combination of a polyelectrolyte having amino functionality and a polyelectrolyte having sulfonic functionality, characterized in that said nanoparticle comprises an outer layer of albumin. A further embodiment is a gold nanoparticle coated with one single layer of said polyelectrolyte having amino or sulfonic functionality, characterized in that said nanoparticle comprises an outer layer of albumin.

Another object of the present invention is the use as a medicament of said nanoparticle, especially against neurodegenerative diseases, most notably neurodegenerative diseases caused by accumulation of protein aggregates in the central nervous system, preferably prion diseases, Alzheimer's disease, Parkinson's disease, Huntington's disease and amyotrophic lateral sclerosis.

Contrary to what is described by the state of the art, see the aforementioned DE 10 2004 040 119, the present inventors have observed that the nanoparticles according to the present invention, when added to the growth medium for cells, of ionic strength comparable to physiological environment, exert their effect without being significantly influenced by the ionic strength of the medium. This aspect represents a technical advantage since it eliminates a critical parameter.

Another object of the present invention is a pharmaceutical composition comprising a therapeutically effective amount of the above particles. These compositions are generally intended for human and veterinary use.

Another object of the present invention is a method for treating a subject affected by a neurodegenerative disease, comprising administering to said subject a therapeutical amount of the above nanoparticle, preferably in the form of a pharmaceutical composition. In a particular aspect of said method, said neurodegenerative disease is caused by protein aggregates. In a more particular aspect, said disease is selected from the group consisting of prion diseases, Alzheimer's disease, Parkinson's disease, Huntington's disease and amyotrophic lateral sclerosis.

Another object of the present invention is the above nanoparticle for use as carrier for a medicament intended to cross the blood-brain barrier, in particular in a human being.

These and other objects of the invention will now be described in detail also by means of figures and examples.

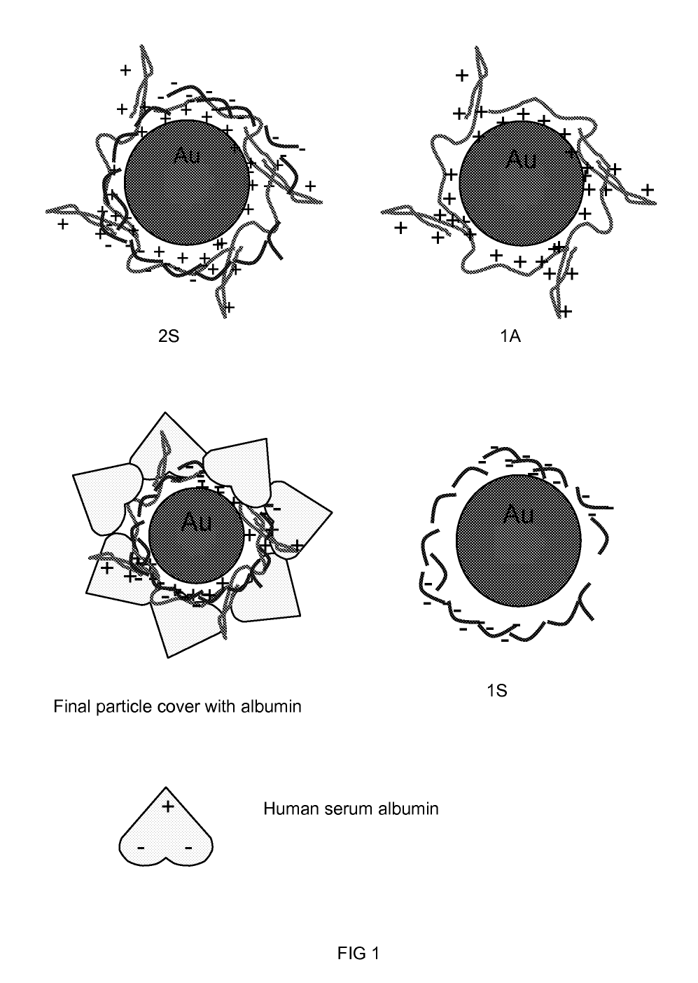

FIG. 1: shows in a schematic way an exemplary structure of nanoparticles according to the present invention, [symbol] shows polystyrenesulfonate (PSS—4.3 kDa, short chain, 23-mer, 23 negative charges), [symbol] indicates polyallylamine hydrochloride (PAH—15 kDa, long chain, 259-mer, 259 positive charges), the heart-shaped symbol means human serum albumin (HSA); 2S indicates two layers of polyelectrolyte, with PSS outer layer; 1A indicates one layer of PAH polyelectrolyte, 1S indicates one layer of PSS polyelectrolyte, and as example it is shown the final preparation of a 2A particle with the last protective layer of albumin.

FIG. 2: shows the cytotoxicity of some particles exemplifying the present invention towards the cells of the blood-brain barrier (modified from Chanana et al., cited above); 2A means particle with (PSS/PAH) in two layers with PAH final, 4A means particle with (PSS/PAH)₂ in four layers and PAH final, 3S particle means particle with (PSS/PAH/PSS) in three layers and PSS final, 5S means particle with (PSS/PAH)₂/PSS in five layers and PSS final.

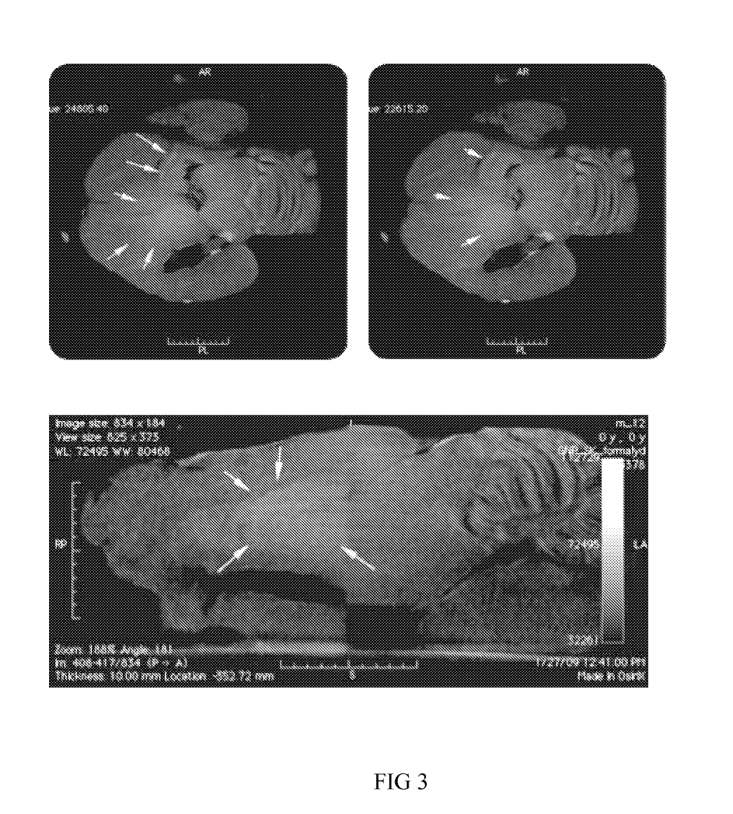

FIG. 3: Tomographic reconstructions of the brain. In the transverse plane (upper panel) and in the sagittal plane (lower panel) of the animal sacrificed after 19 hours. The two transverse planes in the upper panel are separated by 200 μm.

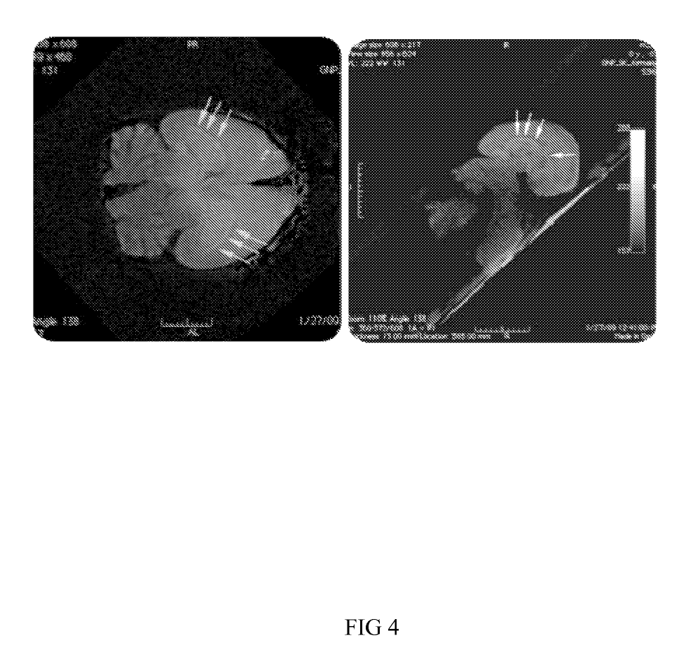

FIG. 4: Tomographic reconstructions of the brain. In the transverse plane (left panel) and in the sagittal plane (right panel) of the animal sacrificed 1 week after particle injection.

### DETAILED DESCRIPTION OF THE INVENTION

The polyelectrolyte with amino functionality preferably is polyallylamine.

The polyelectrolyte having a sulfonic functionality preferably is polystyrenesulfonic.

The polyelectrolyte with amino functionality and the polyelectrolyte having a sulfonic functionality are preferably in the form of a pharmaceutically acceptable salt.

A preferred example of polyelectrolyte with amino functionality is a pharmaceutically acceptable salt of polyallylamine, such as the hydrochloride (PAH).

A preferred example of polyelectrolyte having a sulfonic functionality is a pharmaceutically acceptable salt of polystyrenesulfonate, such as the sodium salt (PSS).

Pharmaceutically acceptable salts are well known to experts in the field and do not require further explanation. See for example Wermuth, C. G. e Stahl, P. H. (eds.) Handbook of Pharmaceutical Salts, Properties; Selection and Use; Verlag Helvetica Chimica Acta, Zurich, 2002.

A preferred example of albumin is human serum albumin (HSA).

The nanoparticles used in the present invention are analogous to those described in the aforementioned works of Schneider and Decher (Nano Letters, 2004, Vol. 4, No. 10, 1833-1839), Dorris et al. (Langmuir, 2008, 24(6), 2532-2538), and Schneider and Decher (Langmuir, 2008, 24, 1778-1789). Particles whose first layer is made of sodium polystyrenesulfonate are described in the aforementioned Chanana et al.

In a preferred embodiment of the present invention, the polyelectrolyte with amine functionality is the polyallylamine hydrochloride (PAH), the polyelectrolyte with sulfonic functionality is sodium polystyrenesulfonate (PSS).

For the purposes of the present invention, particles identical to those described in these works can also be used, except to provide them with an outer layer of albumin, preferably human.

The method of preparation of the particles is the one called LBL, see the above references, for the deposition of layers by means of electrostatic attraction. Initially the polyelectrolyte self assembles on the core of gold.

The molecules of the second layer are attracted by opposite charges of the first layer, while the charges of the core repulse them because of the same charge, as known from LBL theory (see Decher, G.; Polyelectrolyte multilayers, an Overview. In Multilayer thin films; Decher, G., Schlenoff, J., Eds; Wiley-VCH, Weinheim, 2003; p. 1-17.). Moreover, the polycations and polyanions in the so-called precursor layers interpenetrate and this effect of interpenetration is used in the system of the invention with the aim of having a random ratio of sulfonate and amino groups on the surface of final particles (of course in the case of two or more layers of polyelectrolyte) without having to use block copolymers containing the two functionalities of interest.

The outer layer of albumin is essential for the nanoparticle passage and protection of the blood-brain barrier. Preferably human albumin is used, if the nanoparticle is intended for administration to humans, and the preparation is made according to known methods; for flat surfaces, see Glomm W R, Halskau Ø Jr, Hanneseth A M, Volden S. Adsorption behavior of acidic and basic proteins onto citrate-coated Au surfaces correlated to their native fold, stability, and pl. J. Phys. Chem. B. 2007 27; 111(51):14329-45. For gold particles, see: Teichroeb J H, Forrest J A, Jones L W. Size-dependent denaturing kinetics of bovine serum albumin adsorbed onto gold nanospheres. Eur. Phys. J. E. Soft Matter. 2008 August; 26(4):411-5.

However, in the preparation of nanoparticles according to the present invention, difficulties have been met in preparing the outer layer of albumin. Following known methods, there was a phenomenon of aggregation.

The present inventors have developed a new protocol of co-adsorption of the last layer of polyelectrolyte and albumin. In this way, the problem of aggregation is resolved. The method according to the present invention provides dripping a solution of gold nanoparticles, on which the system of polyelectrolytes has already been built by using the LBL technique, in a solution of albumin and the last polyelectrolyte expected.

Therefore, it is another object of the present invention a process for the preparation of nanoparticles herein described comprising the steps of:
a. deposition of polyelectrolyte or polyelectrolytes by means of the “layer-by-layer” technique (LBL);
b. co-adsorption of the last polyelectrolyte and albumin.

In general terms, the preparation of gold particles provides their stabilization with citrate, as disclosed in Turkevich, J.; Stevenson, P. C.; Hillier, J.; A study of the nucleation and growth processes in the synthesis of colloidal gold. Disc. Farad. Soc. 1951, 11, 55-75. Gold nanoparticles have a size higher than 10 nm and lower than 100 nm. Preferably, gold particles have a diameter of 10 nm to 50 nm. As well-known, they can be prepared from a gold derivative, such as NaAuCl₄, and a citrate solution, for example a 1% solution. Citrate solution is quickly added to the boiling gold derivative solution and the mixture is kept boiling for a suitable time. The solution is then allowed to cool at room temperature and stored in dark bottles until subsequent use.

The stabilized nanoparticles are incubated for a suitable time in a solution of the first polyelectrolyte, for example the one with amine function, preferably PAH, more preferably PAH with MW of 15 kDa, or for example the one with sulfonic function, preferably PSS, more preferably PSS with MW of 4.3 kDa. Pure water is preferably used as reaction medium (for example Milli-Q-grade, 18.2 MΩ/cm²). After incubation with the solution of polyelectrolyte, the particle suspension is centrifuged, for example for 20 min at 20.000×g, the supernatant is removed and the particles are resuspended in pure water. Washing is repeated as often as suitable; twice can be sufficient. Thus, the particles coated with the polyelectrolyte are incubated with the polyelectrolyte of opposite charge. In this way, nanoparticles are prepared with 1 to 5 layers of polyelectrolyte, whose outer layer is, by choice, positively charged or negatively charged.

The outer layer of albumin is applied by co-adsorption with the last polyelectrolyte of choice, preferably at pH 7.4. The polyelectrolyte and albumin are added drop by drop and under continuous vortexing to a solution of nanoparticles coated with one or more layers of alternating negatively or positively charged polyelectrolytes. All solutions are prepared in water, preferably at pH 7.4. Washing phases are made with pure water (such as MilliQ water, preferably at pH 7.4). The particles are finally concentrated by centrifugation, such as at 10,000 rpm, for a suitable time.

If these particles are added to the culture medium of neuronal cell lines expressing the prion protein, and replicate the infectious agent, the prion replication is inhibited as a function of concentration. In general terms, particles can be used in a concentration between 5 and 1280 pM. Coated nanoparticle hydrodynamic size is between 28 and 68 nm for those with PSS as the last layer and between 73 and 79 nm for those with PAH. The surface charge is between 52 and 65 mV for positive capsules and between −44 and −56 mV for the negative ones. The extreme effectiveness of the particles used has been demonstrated by the absence of signal in immunoreactivity assays of prion protein resistant to protease digest. This assay is generally accepted as a diagnostic indication of the presence of prion infection.

The particles were examined also for cytotoxicity against the same neurons. The cytotoxicity of PAH is known for different types of cells, while the PSS is considered relatively harmless for the cells (see Chanana et al., cited above).

The particles used according to the present invention clearly show that the PAH is not cytotoxic to the neurons at the concentrations used, while the PSS has shown weak cytotoxicity, around 20% dead cells at higher concentrations.

The nanoparticles described in the present invention can be formulated in appropriate pharmaceutical compositions for human and animal administration.

The preparation of pharmaceutical compositions falls within the normal capacity of technician with ordinary experience in the field and requires no particular description. We can mention as a general reference Remington's Pharmaceutical Sciences, latest edition, Mack Publishing and Co. Additional examples can be found in WO 2008/115854, WO 2008/124131, WO 2008/054544 and WO 2008/021368. Injectable formulations are preferred.

The following examples further illustrate the invention.

### Example 1

### Preparation of the Nanoparticles

Gold particles stabilized with citrate (Turkevich, J.; Stevenson, P. C.; Hillier, J.; A study of the nucleation and growth processes in the synthesis of colloidal gold. Disc. Farad. Soc. 1951, 11, 55-75) having a diameter of 15±1 nm were prepared from 5.3 mg NaAuCl₄ in 25 ml of water boiling under reflux. 1 ml of 1% citrate solution was quickly added and the solution kept boiling for other 20 minutes. The solution was then allowed to cool at room temperature and stored in dark bottles until subsequent use.

Stabilized nanoparticles were then added dropwise in a solution of 3 mg/ml of PAH (MW 15 kDa) or in a solution of 10 mg/ml of PSS (4.3 kDa) prepared with pure water (Milli-Q-grade, 18.2 MΩ/cm²) and then incubated for 20 min. After incubation with the solution of polyelectrolyte, the particle suspension was centrifuged for 20 min at 20.000×g, the supernatant was removed and the particles, which appear as a red gel-like pellet, are resuspended in pure water. Washing is repeated twice. Thus, the particles coated with the polyelectrolyte are incubated with the polyelectrolyte of opposite charge. In this way, nanoparticles are prepared with 1 to 5 layers of polyelectrolyte, whose outer layer is, by choice, positively charged or negatively charged.

### Example 2

Similarly to Example 1, gold nanoparticles of 46 nm in diameter were prepared, from 10.6 mg of NaAuCl₄ in 25 ml of water and fast addition of 750 μl of a 1% citrate solution.

### Example 3

### Cytotoxicity in Neuronal Cell Lines and Tests of Functionality

The number of layers, as well as surface charge, influences ScGT1 cell survival (mouse hypothalamus infected with scrapie) and the concentration at which a complete inhibition of the infectious process can be observed. The same experiments were repeated with ScN2a cells (mouse N2a neuroblastoma infected with scrapie).

The cytotoxicity was determined by counting the ScGT1 cells which survived after incubated for 5 days, stained with calcein-AM in a fluorescence plate reader. For these experiments, the cells were grown in 96-well plates to a density of 25,000 cells/well.

For the functionality test, the preparation was added in different concentrations to the cells and these were grown for 5 days. Thus, the PrP^{Sc} (prion protein from scrapie) was extracted and quantified (100 μg), and digestion was performed with 2 μg of PK (Proteinase K), which is the standard test for the presence of protein aggregates whose form with the incorrect folding (“misfolded”) is resistant to digestion. The resulting solution was analyzed by Western blot, SDS-PAGE gel electrophoresis and the PrP^{Sc} was again quantified with an ELISA assay.

The summary data of different preparations exemplified are shown in the following Tables 1-2.

TABLE 1
PrP^{Sc} inhibition and cellular toxicity of nanoparticles in
ScGT1 and ScN2a cells.

| Particles | | | | |
|---|---|---|---|---|
| Positive surface charge | Prion inhibition | | Cytotoxicity | |
| (polyallylamine (PAH) | ScGT1 | ScN2a | ScGT1 | ScN2a |
| outer layer), diameter | (EC₅₀, | (EC₅₀, | (vital | (vital |
| 15 nm nanogold (NG) | pM) | pM) | cells %) | cells %) |
| 1A | 10 | 10 | 100 | 100 |
| 2A | 10* | 30* | 100 | 97 |
| 3A | 10 | 20 | 100 | 96 |
| 4A | 25 | 25 | 100 | 100 |
| 5A | 20 | 30 | 100 | 92 |
| 2A- | 10* | 30* | 100 | 94 |
| diameter 46 nm | | | | |
| Particles | | | | |
| Negative surface charge | Prion inhibition | | Cytotoxicity | |
| (polystyrenesulfonate | ScGT1 | ScN2a | ScGT1 | ScN2a |
| (PSS) outer layer), | (EC₅₀, | (EC₅₀, | (vital | (vital |
| nanoparticle diameter 15 nm | pM) | pM) | cells %) | cells %) |
| 1S | 150 | 310 | 95 | 92 |
| 2S | 100 | 220 | 97 | 87 |
| 3S | 70 | 150 | 74 | 90 |
| 4S | 50 | 130 | 100 | 90 |
| 5S | 35 | 130 | 84 | 93 |
| 5S- | 90 | 320 | 90 | 91 |
| diameter 46 nm | | | | |

*at the EC₅₀ value

TABLE 2
PrP^{Sc} inhibition and cellular toxicity of quinacrine, imipramine
and nanoparticles in ScGT1 and ScN2a cells.

| | | | % cell | |
|---|---|---|---|---|
| | PrP^{Sc} | | viability ± | |
| Compounds | inhibition^{(a)} | | SEM^{(b)} | |
| | ScGT1 | ScN2a | | |
| | (EC₅₀ ± | (EC₅₀ ± | | |
| Small molecules | SEM, μM) | SEM, μM) | ScGT1 | ScN2a |
| Quinacrine | 0.4 ± 0.1 | 0.3 ± 0.1 | 100 ± 4 | 100 ± 2 |
| Imipramime | 6.2 ± 0.4 | 5.5 ± 0.5 | 100 ± 7 | 100 ± 5 |
| | ScGT1 | ScN2a | | |
| | (EC₅₀ ± | (EC₅₀ ± | | |
| Nanoparticles | SEM, pM) | SEM, pM) | ScGT1 | ScN2a |
| Positive surface | | | | |
| charge-PAH | | | | |
| (NG-15 nm) | | | | |
| 1A | 8.3 ± 0.5 | 8.4 ± 0.6 | 100 ± 6 | 100 ± 3 |
| 2A | 8.8 ± 0.2 | 24.5 ± 1.0 | 100 ± 1 | 97 ± 1 |
| 3A | 10.1 ± 0.2 | 20.4 ± 0.5 | 100 ± 7 | 96 ± 3 |
| 4A | 25.4 ± 1.3 | 25.1 ± 1.2 | 100 ± 6 | 100 ± 5 |
| 5A | 20.1 ± 1.1 | 30.0 ± 1.4 | 100 ± 3 | 92 ± 1 |
| Negative surface | | | | |
| charge -PSS | | | | |
| (NG-15 nm) | | | | |
| 1S | 121.4 ± 6.5 | 248.7 ± 12.9 | 95 ± 2 | 92 ± 5 |
| 2S | 99.8 ± 4.7 | 220.3 ± 11.8 | 97 ± 1 | 87 ± 3 |
| 3S | 70.1 ± 3.2 | 149.5 ± 6.1 | 74 ± 7 | 90 ± 3 |
| 4S | 50.3 ± 2.0 | 130.1 ± 5.4 | 100 ± 2 | 90 ± 7 |
| 5S | 35.0 ± 1.4 | 129.9 ± 7.1 | 84 ± 8 | 93 ± 4 |
| NG-46 nm | | | | |
| 2A | 10.3 ± 0.3 | 30.2 ± 1.7 | 100 ± 4 | 94 ± 2 |
| 5S | 89.7 ± 3.5 | 329.5 ± 10.7 | 90 ± 1 | 91 ± 6 |

^{(a)}Compound concentration required to reduce 50% PrP^{Sc} level versus untreated cells.
^{(b)}Cell viability at EC₅₀ values was determined by calcein-AM cytotoxicity assay and expressed as an average percentage of viable cells versus untreated control cells.

Standard errors from three experiments are given.

The relative potency of known drugs, such as quinacrine and imipramine, was used as a control for anti-prion activity in the cell models. Table 2 indicates the potency of either quinacrine or imipramine to be similar to previous publications, namely EC₅₀ of quinacrine was 0.4±0.1 and 0.3±0.1 μM for ScGT1 and ScN2a, respectively; whereas for imipramine EC₅₀ was 6.2±0.4 and 5.5±0.5 μM for ScGT1 and ScN2a, respectively. As a control, citrate stabilized gold particles without polyelectrolyte layers did not show any detectable prion inhibitory activity.

The number of layers, in addition to the surface charge of the outermost layer for the coated nanoparticles, influenced the neuronal ScGT1 and ScN2a cell survival, as shown in Table 2. Cytotoxicity was determined by measuring cell survival after incubation in the drug-doped medium for 5 days, and assayed by the calcein-AM assay. For these experiments, which were carried out in 96 well-plates, cells were grown from a starting density of 25,000 ScGT1 cells and 30,000 ScN2a cells per well. The cell viability was between 92-100% for positively charged particles (1-5A), and 74-100% for negatively charged particles (1-5 S) (Table 2).

The concentration at which a complete inhibition of PrP^{Sc} formation in ScGT1 and ScN2a cells took place was determined from the SDS-PAGE gels. Particle preparations were added at different concentrations to scrapie-infected cells, and the inhibitory activity was measured over 5 days. PrP^{Sc} levels were quantified either by western blot or by ELISA. The resulting EC₅₀ of the particles with a positive outermost layer (mA) were in the range of 8.3±0.5-25.4±1.3 pM in ScGT1 and 8.4±0.6-30.0±1.4 pM in ScN2a cells (Table 2). In both cases, the influence of size and number of layers on efficacy is limited. However, prion inhibition by particles with a negative outermost layer (nS) showed an increase in efficacy with a higher number of layers. In particular, EC₅₀ of 15 was 121.4±6.5 pM and 5S was 35.0±1.4 pM in ScGT1 whereas the EC₅₀ of 15 was 248.7±12.9 pM and 5S was 129.9±7.1 pM in ScN2a cells (Table 2).

In order to investigate a possible influence of the particle curvature on the efficacy of prion inhibition, a larger-sized gold particle, 46 nm was used. The present inventors tested only for the most effective 2A and 5S coatings. Both cell lines showed no variation in cell viability (Table 2), which was in the range of 90-100%, and therefore was not different when compared to the smaller nanogold particle (15 nm). Prion inhibition of 2A −46 nm was similar to 2A, and 5S −46 nm was 3 times less effective than 5S. The results of 2A −46 and 5S −46 nm are shown in Table 2.

Since potent anti-prion activity for 2A and 5S was found in scrapie-infected cells, these two particles were chosen to test their ability to inhibit recombinant PrP fibril formation in amyloid seeding assay (ASA) (Table 3).

TABLE 3
Effect ofthe nanoparticles on fibril formation and ASA

| | M5 (lag | Gemini EM (lag |
|---|---|---|
| Assay | phase, hours)* | phase, hours)* |
| PrP | 30-35 | 50-55 |
| PrP + 2A | 45-50 | 55-60 |
| PrP + 5S | 40-45 | 55-60 |
| PrP-ScN2a | 25-30 | 35-40 |
| PrP-ScN2a + 2A | 35-40 | 45-50 |
| PrP-ScN2a + 5S | 30-35 | 50-55 |
| PrP-ScGT1 | 25-30 | 40-45 |
| PrP-ScGT1 + 2A | 30-35 | 50-55 |
| PrP-ScGT1 + 5S | 25-30 | 50-55 |

*Lag phase of amyloid-formation kinetics are compared between SpectraMax M5 and Gemini EM instruments (Molecular Devices) in the assays using full-length MoPrP(23-230) and amyloid seeding with ScN2a- and ScGT1-PTA precipitated protein in presence of coated gold. Fifty pM of 2A coated nanogold or 200 pM of 5S coated nanogold was added to each well.

The Student's t-test (two-tailed) was used to determine significant differences among measurements. For the M5 p<0.05 (n=4); for the Gemini EM p<0.01 (n=4). In Table 3, 50 pM and 200 pM of both 2A and 5S delayed significantly fibril formation as expressed by the lag phase of the amyloid-formation kinetics. Moreover, 2A and 5S extended the lag phase by 5-15 hours, therefore showing a much slower kinetics than the control. From the potency of 2A and 5S in delaying of PrP fibril formation, it is suggested that these nanoparticles may interact directly with PrP to prevent PrP^{C} conversion to a pathogenic PrP^{Sc}-like form. In standard recombinant PrP fibril formation assays using MoPrP (23-230) alone, both 2A and 5S exhibited PrP amyloid fibril forming inhibitory activity. In addition, in ASA, PrP^{Sc} proteins from PTA-precipitated ScGT1 or ScN2a cell extracts were able to seed amyloid PrP formation and to promote fibril formation exhibiting much shorter lag phase kinetics. On the contrary, in the presence of nanoparticles, the lag phase of amyloid PrP formation was extended significantly in ASA.

### Example 4

Biodistribution In Vivo

The nanoparticles according to the present invention must have an outer layer of albumin to be administered to the animal and cross the blood brain barrier. In this example, human albumin was used. The layer of albumin was applied by co-adsorption with PAH at pH 7.4. 500 μl of PAH (1 mg/ml) and 500 μl of human serum albumin (HSA) were added dropwise and under continuous vortexing to a solution of gold nanoparticles coated with 1 layer of sodium polystyrenesulfonate (PSS) and named 1S. All solutions were prepared in water at pH 7.4. Washings were made with MilliQ water at pH 7.4. In this environment, the coated particles have a hydrodynamic diameter of 90±2 nm in dynamic light scattering and a surface charge of 36±1 mV in zeta potential measurements. The particles were concentrated 15 times to a final volume of 200 μl by centrifugation at 10,000 rpm for 40 min, then 100 μl of a Ringer's solution were added. Approximately 150-200 μl of solution were injected into the tail vein of healthy C57 black mice, under gas anesthesia. The coating was prepared the same day of injection in mouse. The particles injected in the Ringer's solution show a hydrodynamic radius of 134±2 nm in a dynamic light scattering and a surface charge of 31±1 mV in zeta potential measurements. With the purpose of revelation of biodistribution, both the polyallylamine and albumin have been covalently labeled with cy5.5, a dye that can be displayed in the preclinical image analyzer eXplore Optix with a excitation wavelength of 670 nm and an emission wavelength of 700 nm. NIR (near infrared) light allows a deep penetration into the tissue and a low background signal. The instrument is capable of detecting a fluorescence signal 5-9 mm below the phantom surface and therefore allows the visualization of molecules labeled with the dye in the brain or other organs.

The biodistribution was followed for 10 days after injection. The particles begin to accumulate in the brain 15 minutes after injection and the concentration increases up to 24 hours. The mice administered with nanoparticles in accordance with the present invention showed no change in behavior or other signs of damage of blood-brain barrier, which allows the conclusion that the cytotoxicity of the nanoparticles of the present invention is either decreased or completely absent. Cytotoxicity studies show that PAH (polyallylamine hydrochloride) is slightly less cytotoxic towards neurons compared to PSS (polystyrenesulfonate) at the concentrations used in cell culture. This behavior is opposite to that towards the blood-brain barrier endothelial cells, which are mainly damaged by PAH compared to PSS. This result is totally unexpected, since PAH is known for its cytotoxicity towards many other cell types (Boussif, O.; Delair, T.; Brua, C.; Veron, L.; Pavirani, A.; Kolbe, H. V. J., Synthesis of Polyallylamine Derivatives and Their Use as Gene Transfer Vectors in Vitro. Bioconjugate Chem. 1999, 10, 877-883).

For both cell types tested (ScGT1 and ScN2a) the EC₅₀ (inhibition of 50% of prion aggregation) was determined. Cytotoxicity is determined by staining with calcein-AM (Calcein acetoxymethyl ester, a fluorescent compound that permeates the cells and is converted by cellular esterases into calcein, the anionic fluorescent form). None of the preparations tested shows a survival 80% lower than EC₅₀ values. In order to study whether the curvature of the particle has some influence on cytotoxicity or on prion inhibition, particles of larger diameter were also tested for the more effectively preparation with particles of size of 15 nm (46 nm, 2A and 5S).

In general, it can be said that ScN2a are less affected by coated particles by a factor of 3 compared to ScGT1. For preparations with a positive outer layer (indicated with the symbol mA, where m is the total number of layers and A indicates PAH) the EC₅₀ is 14±7 pM for ScGT1 and 24±8 pM for ScN2a. The influence of size and number of layers is negligible in both cases. Cell viability is between 92 and 100%. This is in contrast with the data of the particles with the outer layer of sulfonate (indicated with the symbol nS, where n is the total number of layers and S indicates PSS). In this case, the effectiveness of prion inhibition increases with the number of layers. In the case of ScGT1, 5S is 50 times more effective than 1S, whereas in ScN2a case it has a double efficacy. Comparing the curves of the particles and the average size, it was seen that the larger particles (46 nm) are 3 times less effective than small ones (15 nm). As previously mentioned, the cytotoxicity is higher at the concentration of EC₅₀ for the negative outer layer.

One of the drawbacks of NIR-TD imaging is that it has a limit resolution of 0.5 mm. Therefore, it is not possible to clearly assess the specific localization of the nanoparticles inside of the brain.

In order to localize the coated particles more precisely in the brain and solve the question if they cross the BBB it was used X-ray microtomography, confocal laser scanning microscopy (CLSM), and fluorescence images with two different staining techniques. To ascertain that the particles we observed in NIR-TD are the same like we visualize in CLSM we used a double staining of the particles, covalently labeling HSA with cy5.5 and PAH with FITC (fluorescein isothiocyanate).

X-Ray microtomographic results of two treated mice are shown in FIG. 3 (19 h) and in FIG. 4 (7 days). The data reveal two interesting findings. Firstly, there is good contrast between the brain's white and grey matter. This is prominent in the rat's cerebellum. Although such differentiation is rarely possible with absorption-based X-ray CT scans, phase contrast radiography clearly resolves these small density differences between the two brain tissues. Secondly, as indicated by the arrows in the reconstructed transverse and sagittal planes higher contrast and subsequently higher particle concentration is recognized in regions of the thalamus and hypothalamus. For the animal sacrificed after 19 hours (FIG. 3) an area contrast comprising the entire region of the thalamus/hypothalamus is apparent. In addition a thin line of higher absorption at the boundary of the thalamus is visible indicating an accumulation of the gold nanoparticles. In the animal sacrificed 7 days after nanoparticle injection the area contrast in the thalamus/hypothalamus complex disappeared but now two thin lines of higher absorption/contrast are prominent (indicated by the arrows in FIG. 4) indicating an accumulation of gold on the boundary of lamellae separating different thalamic subparts.

Microtome brain sections were imaged in more detail by means of CLSM, in order to localize the coated nanoparticles on a cellular level. The fluorescent images confirmed the observation with x-ray that the nanoparticles accumulated mainly in the thalamus zone, hippocampus and also in the cortex. The direct visualization of the FITC-labeled nanoparticles was difficult due to the significant autofluorescence of the tissue in the same range. Therefore, a spectral analysis of the emission signal was performed. The spectra allowed us to distinguish the autofluorescence signal from the FITC signal. The presence of the nanoparticles in the brain tissue was confirmed and a high-resolution localization down to cell level was possible. With CLSM images an evenly distributed pattern of fluorescent spots in the brain tissue could be detected, indicating that the nanoparticles crossed the BBB.

Localization and cellular distribution pattern of nanoparticles of the sectioned brain tissue was visualized with a combination of three different staining techniques (Nissl, DAPI, and FITC staining). DAPI fluorescence marks selectively the nuclei in blue while the Nissl stain allows us to visualize the cell body of both, neurons and glia. The nanoparticles are emitting green fluorescence. The stained brain sections were visualized with a combination of bright field white light and epifluorescence microscope using different emission filters.

The images acquired with the different filters were merged. The regions of the brain, where high nanoparticle concentrations were detected with X-ray and CLMS, were analyzed in more detail for sub cellular nanoparticle distribution. Small bright green dots, related to the coated nanoparticles labeled with FITC, were observed on the cellular surface but also in the cell interior of specific brain cells stained with Nissl. With the DAPI staining no co-localization could be detected and therefore entrance in the nucleus can be excluded.

In the hippocampus zone, accumulation of particles inside of dentate granule cells (pyramidal cells of CA1 and CA3 region) as well in the granule cell layer of the dentate gyrus was observed. The thalamus was the brain region where it was observed a higher accumulation of particles, also the cortex showed high concentration of nanoparticles inside of the cells. No significant amount of nanoparticles was detected in the cerebellum. However, particle accumulation was found inside of the Purkinje cells, which are a class of GABAergic neurons located in the cerebellar cortex. Moreover nanoparticles were also localized in the medulla towards the spinal cord.

With these different staining techniques it can be concluded, that the nanoparticles accumulate in specific neuronal cells in different brain regions and that they are internalized in the cells but do not enter the nucleus. The regions were the particles were visualized on a cellular level are essentially the same like the ones found in CLSM and x-ray tomography.

For the first time we were able to show that not only the coated nanogold particles were entering the brain by passing the blood-brain barrier and also that they are not homogenously distributed in the brain liquor but they are taken selectively by some cells in different regions of the brain. The cells were identified and the biodistribution in mice was followed starting from the whole body down to sub cellular distribution in the specific cells.

Another important aspect from this invention is the fact that, with this nanoparticle system it is possible to transport a wide range drugs and due to their accumulation into specific cells it is possible to target specific brain disorders reducing like this unwanted side effects.

1. Gold nanoparticle coated with from two to five layers of a combination of a polyelectrolyte having amino functionality and a polyelectrolyte having sulfonic functionality, or with one single layer of said polyelectrolyte having amino or sulfonic functionality, wherein said gold nanoparticle has a size higher than 10 nm and lower than 100 nm, characterized in that said nanoparticle comprises an outer layer of albumin.

2. Nanoparticle according to claim 1, wherein said polyelectrolyte is in the form of a pharmaceutically acceptable salt.

3. Nanoparticle according to claim 1, wherein said polyelectrolyte having amine functionality is polyallylamine.

4. Nanoparticle according to claim 1, wherein said polyelectrolyte having sulfonic functionality is polystyrenesulfonic.

5. Nanoparticle according to claim 1, wherein said albumin is human serum albumin.

6. Process for the preparation of the nanoparticles of claim 1, comprising the steps of:
a. deposition of the polyelectrolyte or the polyelectrolytes by means of the layer-by-layer technique (LBL);
b. co-adsorption of the final polyelectrolyte and albumin.

7. The nanoparticle of claim 1 for use as medicament.

8. The nanoparticle according to claim 7, wherein said medicament is for the treatment of neurodegenerative diseases.

9. The nanoparticle according to claim 8, wherein said diseases are caused by protein aggregates.

10. The nanoparticle according to claim 8, wherein said disease is selected from the group consisting of prion diseases, Alzheimer's disease, Parkinson's disease, Huntington's disease and amyotrophic lateral sclerosis.

11. The nanoparticle of claim 1 for use as carriers for a medicament intended to cross blood-brain barrier.

12. Pharmaceutical composition for human or veterinary use comprising an effective amount of nanoparticles of claim 1.
